(19) United States
(12) Patent Application Publication
(10) Pub. No.: US 2024/0074990 A1
(43) Pub. Date: Mar. 7, 2024

(54) APPLICATION OF GOSSYPOL AND ITS OPTICAL ISOMERS TO PREPARATION OF CORONAVIRUS 3CL PROTEASE INHIBITOR

(71) Applicants: SHAANXI PANLONG PHARMACEUTICAL CO ., LTD, Xi'an (CN); XIXIAN NEW DISTRICT FENGHOU ORIGINAL PHARMACEUTICAL TECHNOLOGY CO., LTD, Xi'an (CN)

(72) Inventors: Xiaolin XIE, Xi'an (CN); Dezhu ZHANG, Xi'an (CN); Zhao MA, Xi'an (CN); Boyang LI, Xi'an (CN); Xuhua ZHOU, Xi'an (CN); Lei TIAN, Xi'an (CN); Chengyuan LIANG, Xi'an (CN); Taotao QIANG, Xi'an (CN); Jingyi LI, Xi'an (CN); Liang XIN, Xi'an (CN); Shaojun ZHANG, Xi'an (CN); Kangxiong WU, Xi'an (CN); Xiuding YANG, Xi'an (CN); Sundian LIU, Xi'an (CN); Yuting LIU, Xi'an (CN)

(21) Appl. No.: 18/186,160

(22) Filed: Mar. 18, 2023

(30) Foreign Application Priority Data
Aug. 18, 2022 (CN) 202210994739.3

(51) Int. Cl. A61K 31/12; A61P 31/14

(52) U.S. Cl. CPC A61K 31/12; A61P 31/14

(57) ABSTRACT

The present invention relates to application of gossypol and its optical isomers to preparation of an anti-Coronavirus drug. Specifically disclosed is application of pharmaceutically acceptable salts, solvates, isotopes, stereoisomers, stereoisomer mixtures, and tautomers of the gossypol and its optical isomers to preparation of a drug for preventing and/or treating a disease caused by a Coronavirus. The Coronavirus is MERS-CoV, SARS-CoV, or SARS-CoV-2. It is found in the present invention for the first time that the gossypol and its optical isomers can inhibit the activity of Coronavirus 3CL proteases to achieve an anti-Coronavirus effect. The half maximal inhibitory concentrations of gossypol and (−)-gossypol to SARS-CoV proteases and SARS-CoV-2 3CL proteases are all lower than 10 μM. The anti-Coronavirus 3CL protease effect is good. Therefore, the gossypol and its optical isomers have the potential for use in the preparation of a drug for preventing and/or treating novel Coronavirus infections.

### TECHNICAL FIELD

The present invention belongs to the field of medicine, and in particular to application of gossypol and its optical isomers to preparation of a Coronavirus 3CL protease inhibitor.

### BACKGROUND OF THE INVENTION

Coronaviruses belong to the genus Coronavirus in the family Coronaviridae. Viruses of the genus Coronavirus are enveloped positive-stranded single-stranded RNA viruses of about 80-120 nm in diameter, and their genetic material is the largest of all RNA viruses, which generally infect only vertebrates including humans, rodents, pigs, cats, dogs, and avians. The main Coronaviruses in recent outbreaks are MERS-CoV, SARS-CoV, and SARS-CoV-2. Coronavirus particles are irregular in shape and about 60-220 nm in diameter. The viruses contain about 30,000 bases and 14 open reading frames and can encode replicase, 4 structural proteins (S, E, M, and N), 16 nonstructural proteins, and 9 accessory proteins. NSP is a nonstructural protein of the SARS-CoV-2 virus, which plays an important role in the replication and transcription cycle of the virus. NSP5 is the major protease of SARS-CoV-2, also known as 3CLike protease, and has high structural similarity and conservation (FIG. 1). It can be seen that 3CL^{pro} can be used as a homologous target for the development of transcriptional drugs that inhibit infection and replication of multiple Coronaviruses.

A 3CL protease can process viral polyproteins. Viral RNA is initially translated into polyproteins upon entry into human cells. The 3CL protease can cleave 12 smaller proteins from the polyproteins, which are involved in the replication of viral RNA. Since the 3CL protease is an important protease that play a crucial role in inhibiting viral replication and has no homologous protein in humans, the 3CL protease is an ideal target for antiviral drug development. In addition, the 3CL protease is highly conserved in β Coronaviruses, and the screened 3CL protease inhibitors have a degree of broad-spectrum anti-Coronavirus ability. At present, no small molecule Coronavirus main protease 3CL protease inhibitor with good efficacy and high specificity has been identified.

Gossypol is a yellow polyphenolic compound isolated from the roots, stems, and seeds of cotton, a member of the mallow family, has a variety of biological activities, and is very widely applied in anti-fertility, anti-tumor, and treatment of gynecological diseases. However, currently there are no reports related to the inhibition of 3CL proteases by gossypol and its optical isomers.

### SUMMARY OF THE INVENTION

An objective of the present invention is to provide application of gossypol and its optical isomers to preparation of 3CL protease inhibitors and preparation of an antiviral drug.

Specifically, to resolve the technical problem of the present invention, the following technical solution is used.

The present invention provides application of pharmaceutically acceptable salts, solvates, isotopes, stereoisomers, stereoisomer mixtures, and tautomers of the gossypol and its optical isomers to preparation of a drug for preventing and/or treating a disease caused by a Coronavirus.

In the technical solution of the present invention, the Coronavirus includes: SARS-CoV, MERS-CoV, and SARS-CoV-2.

In the technical solution of the present invention, the disease caused by the Coronavirus is an infectious disease caused by SARS-CoV, MERS-CoV, or SARS-CoV-2 or a complication thereof.

In the technical solution of the present invention, the Coronavirus protease is a 3CL protease.

In the technical solution of the present invention, the structural formulas of gossypol and its optical isomers are as follows:

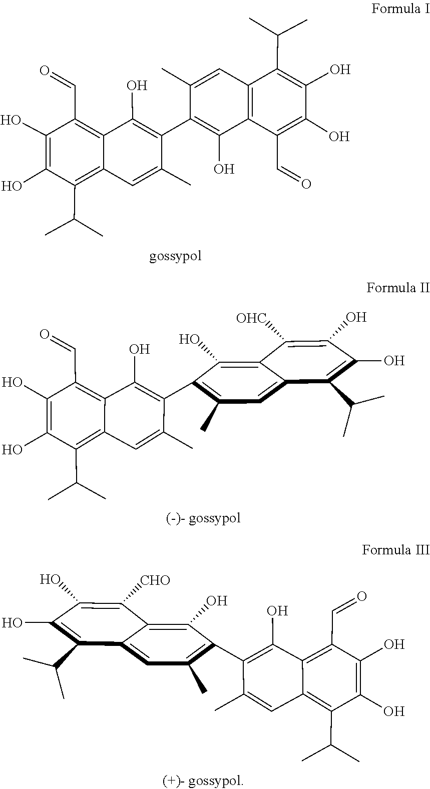

In the technical solution of the present invention, one or more of compounds of gossypol and its optical isomers or pharmaceutically acceptable salts, solvates, isotopes, stereoisomers, stereoisomer mixtures, and tautomers thereof are used as active components for application of preparing a drug for preventing and/or treating a disease caused by a Coronavirus.

Another aspect of the present invention provides a pharmaceutical composition for treating and/or preventing a disease caused by a Coronavirus, including one or more of compounds of gossypol and its optical isomers or pharmaceutically acceptable salts, solvates, isotopes, stereoisomers, stereoisomer mixtures, and tautomers thereof.

In the technical solution of the present invention, dosage forms of the drug and the pharmaceutical composition include: any one of capsules, tablets, granules, gels, slow-release formulations, oral solutions, dripping pills, emulsions, injections, and nanoagents.

The technical solution of the present invention has the following beneficial effects and characteristics:

It is found in the present invention for the first time that the gossypol and its optical isomers can inhibit the activity of 3CL proteases to achieve an anti-Coronavirus effect. The half maximal inhibitory concentration is low, and the activity of 3CL proteases can be significantly inhibited, so that the gossypol and its optical isomers can be used for preventing and/or treating Coronavirus infections, and further used for preparing preventing and/or treating an anti-Coronavirus drug.

### BRIEF DESCRIPTION OF THE DRAWINGS

FIG. 1 shows simulated overlay diagram of MERS-CoV 3CL^{pro} (PDB ID 4RSP, green), SARS-CoV 3CL^{pro} (PDB ID 1Q2W, red), and SARS-CoV-2 3CL^{pro} (PDB ID 6LU7, blue).

FIG. 2 shows the inhibitory effect of gossypol on SARS-CoV 3CL proteases in the present invention;

FIG. 3 shows the inhibitory effect of (−)-gossypol on SARS-CoV 3CL proteases in the present invention;

FIG. 4 shows the inhibitory effect of (+)-gossypol on SARS-CoV 3CL proteases in the present invention;

FIG. 5 shows the inhibitory effect of acetate gossypol on SARS-CoV 3CL proteases in the present invention;

FIG. 6 shows the inhibitory effect of gossypol on SARS-CoV-2 3CL proteases in the present invention;

FIG. 7 shows the inhibitory effect of (−)-gossypol on SARS-CoV-2 3CL proteases in the present invention;

FIG. 8 shows the inhibitory effect of (+)-gossypol on SARS-CoV-2 3CL proteases in the present invention; and

FIG. 9 shows the inhibitory effect of acetate gossypol on SARS-CoV-2 3CL proteases in the present invention.

### DETAILED DESCRIPTION OF EMBODIMENTS

To enable a person skilled in the art to better understand the solutions of the present invention, the following clearly and completely describes the technical solutions in embodiments of the present invention with reference to the accompanying drawings in embodiments of the present invention. Apparently, the described embodiments are some rather than all of the embodiments of the present invention. All other embodiments obtained by persons of ordinary skill in the art based on the embodiments of the present invention without creative efforts shall fall within the protection scope of the present invention.

It needs to be noted that the terms such as “first” and “second” in the specification, claims, and the foregoing accompanying drawings of the present invention are only used to distinguish between similar objects, but are not used to describe a specific order or time sequence. It should be understood that the data thus used are interchangeable in appropriate circumstances and that the embodiments of the present invention described herein can be implemented in other sequences than those illustrated or described. In addition, the terms “include”, “comprise”, and any variation of such terms in in the description and claims of the present invention is intended to cover a non-exclusive inclusion. For example, a process, method, system, product or device that includes a series of steps or units not only includes those steps or units specified expressly, but also includes other steps or units that are not specified expressly or are inherent to the process, method, product or device.

This application is described below in detail with reference to specific embodiments.

The following raw material sources are exemplary descriptions.
gossypol (CAS: 303-45-7), (−)-gossypol (CAS: 90141-22-3), (+)-gossypol (CAS: 20300-26-9), and acetate gossypol are purchased from Chengdu Yirui Biotechnology Co., Ltd.

Embodiment 1: In-vitro enzyme activity inhibit test of gossypol, (−)-gossypol, (+)-gossypol, and acetate gossypol on SARS-CoV 3CL proteases

A fluorescence resonance energy transfer method is used to evaluate the inhibitory effects of the four compounds on the activity of SARS-CoV 3CL protease (each compound is tested in parallel three times).

An appropriate amount of the foregoing compound is weighed, and is prepared with DMSO into a solution with an appropriate concentration gradient. 5 μL of the prepared solution and 91 μL of Assay Reagent (Assay Buffer: SARS-CoV M^{pro}/3CL^{pro}=90:1, purchased from Shanghai Beyotime Biotechnology Co., Ltd.) are sequentially added to a black 96-well plate and are evenly mixed. After incubation in the dark at 37° C. for 10 minutes. 4 μL of Substrate (100 μM Dabcyl-KTSAVLQSGFRKME-Edans, purchased from Shanghai Beyotime Biotechnology Co., Ltd.) is quickly added to each well, and is evenly mixed. After incubation in the dark at 37° C. for 5 min, the signal tends to be stable. A fluorescence test is performed within 5 min to 30 min by using a multifunctional microplate reader (purchased from Thermo Fisher Scientific Co., Ltd., Varioskan Flash). An inhibition percentage of the sample is calculated. The excitation wavelength is 340 nm, and the emission wavelength is 490 nm. Assay Reagent that does not contain the compound is used for 100% enzyme activity control, Assay Buffer that does not contain SARS-CoV M^{pro}/3CL protease is used for blank control, and the rest has the same processing method. Nonlinear regression analysis is performed by using the software GraphPad Prism to calculate an IC₅₀ value of the sample (gossypol, (−)-gossypol, and (+)-gossypol in the present invention).

$$Inhibition rate ( \% ) = \frac{{RFU}_{100 \% enzyme activity control} - {RFU}_{sample}}{{RFU}_{100 \% enzyme activity control} - {RFU}_{blank control}} \times 100 \%$$

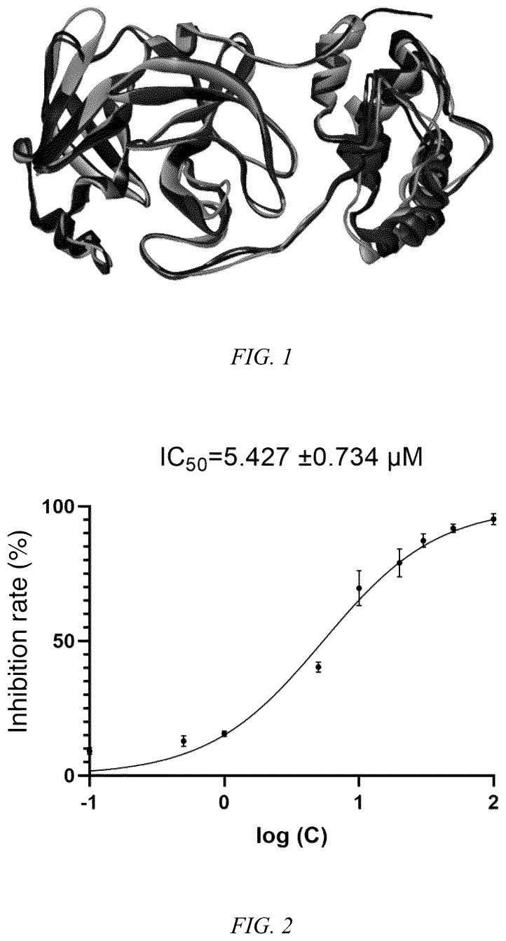

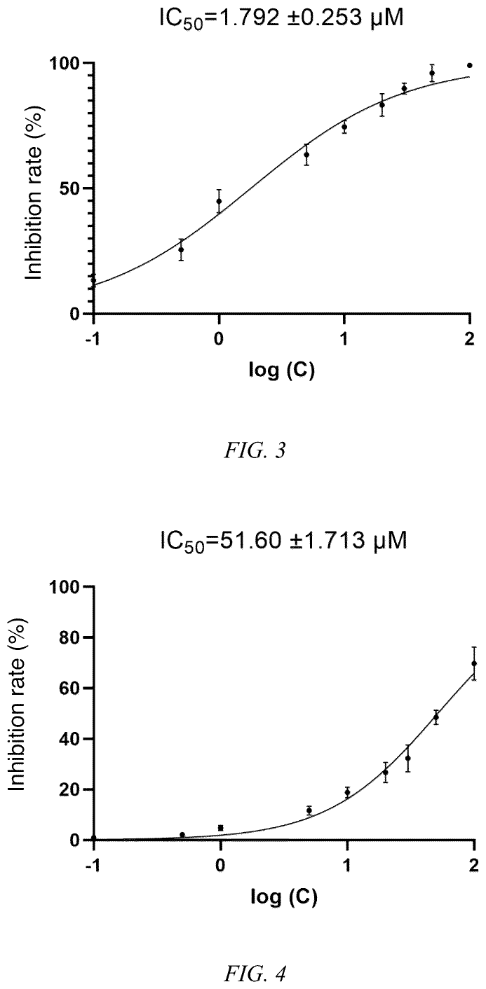

Experimental results are shown in Table 1, the gossypol, (−)-gossypol, (+)-gossypol, and acetate gossypol in this embodiment all have inhibitory activity on SARS-CoV 3CL proteases. The gossypol, (−)-gossypol, and acetate gossypol have a stronger inhibitory effect on SARS-CoV 3CL proteases, and the IC₅₀ values are all below 10 μM. The IC₅₀ value of (−)-gossypol reaches 1.792±0.253 μM (FIG. 1 to FIG. 3 respectively shown the inhibitory effects of gossypol, (−)-gossypol, (+)-gossypol, and acetate gossypol on SARS-CoV 3CL proteases.

TABLE 1
Inhibitory activity of gossypol, (−)- gossypol, (+)-
gossypol, and acetate gossypol on SARS-CoV 3CL proteases

| | Compound | | | | | | | | | | | |
|---|---|---|---|---|---|---|---|---|---|---|---|---|
| | Gossypol (IC₅₀ = | | | (−)- gossypol (IC₅₀ = | | | (+)- gossypol (IC₅₀ = | | | Acetate gossypol (IC₅₀ = | | |
| | 5.427 ± 0.734) | | | 1.792 ± 0.253) | | | 51.60 ± 1.713) | | | 5.157 ± 0.712) | | |
| Concentration/μM | 1 | 2 | 3 | 1 | 2 | 3 | 1 | 2 | 3 | 1 | 2 | 3 |
| 0.1 | 8.21 | 5.92 | 13.30 | 13.11 | 10.83 | 15.87 | 0.33 | 0.98 | 1.44 | 9.43 | 6.51 | 14.17 |
| 0.5 | 13.33 | 9.83 | 20.12 | 24.54 | 21.88 | 30.26 | 1.32 | 2.12 | 2.98 | 14.67 | 10.73 | 21.69 |
| 1 | 25.56 | 18.54 | 31.50 | 43.78 | 40.92 | 50.01 | 3.96 | 4.54 | 5.87 | 26.61 | 19.97 | 32.84 |
| 5 | 40.37 | 35.68 | 44.78 | 64.32 | 58.9 | 67.22 | 11.58 | 9.88 | 13.43 | 41.15 | 36.26 | 45.91 |
| 10 | 61.18 | 51.33 | 64.31 | 77.32 | 72.54 | 73.76 | 19.12 | 16.53 | 20.66 | 62.48 | 52.56 | 65.81 |
| 20 | 74.98 | 64.56 | 77.58 | 86.43 | 78.13 | 85.43 | 22.9 | 26.42 | 30.76 | 75.14 | 65.81 | 78.97 |
| 30 | 85.09 | 75.44 | 86.42 | 91.83 | 87.54 | 90.32 | 28.14 | 30.43 | 38.33 | 86.61 | 76.15 | 87.62 |
| 50 | 92.12 | 84.12 | 93.41 | 98.42 | 92.10 | 97.54 | 45.56 | 48.42 | 51.33 | 93.52 | 85.63 | 94.62 |
| 100 | 96.33 | 90.09 | 98.44 | 99.74 | 98.12 | 99.53 | 72.33 | 62.28 | 74.43 | 97.81 | 91.32 | 98.71 |

Embodiment 2: In-vitro enzyme activity inhibit test of gossypol, (−)-gossypol, (+)-gossypol, and acetate gossypol on SARS-CoV-2 3CL proteases

A fluorescence resonance energy transfer method is used to evaluate the inhibitory effects of the four compounds on the activity of SARS-CoV-2 3CL protease (each compound is tested in parallel three times).

An appropriate amount of the foregoing compound is weighed, and is prepared with DMSO into a solution with an appropriate concentration gradient. 5 μL of the prepared solution and 91 μL of Assay Reagent (Assay Buffer: SARS-CoV-2 M^{pro}/3CL^{pro}=90:1, purchased from Shanghai Beyotime Biotechnology Co., Ltd.) are sequentially added to a black 96-well plate and are evenly mixed. After incubation in the dark at 37° C. for 10 minutes. 4 μL of Substrate (100 μM Dabcyl-KTSAVLQSGFRKME-Edans, purchased from Shanghai Beyotime Biotechnology Co., Ltd.) is quickly added to each well, and is evenly mixed. After incubation in the dark at 37° C. for 5 min, the signal tends to be stable. A fluorescence test is performed within 5 min to 30 min by using a multifunctional microplate reader (purchased from Thermo Fisher Scientific Co., Ltd., Varioskan Flash). An inhibition percentage of the sample is calculated. The excitation wavelength is 340 nm, and the emission wavelength is 490 nm. Assay Reagent that does not contain the compound is used for 100% enzyme activity control, Assay Buffer that does not contain SARS-CoV-2 M^{pro}/3CL protease is used for blank control, and the rest has the same processing method. Nonlinear regression analysis is performed by using the software GraphPad Prism to calculate an IC₅₀ value of the sample (gossypol, (−)-gossypol, and (+)-gossypol in the present invention).

$$Inhibition rate ( \% ) = \frac{{RFU}_{100 \% enzyme activity control} - {RFU}_{sample}}{{RFU}_{100 \% enzyme activity control} - {RFU}_{blank control}} \times 100 \%$$

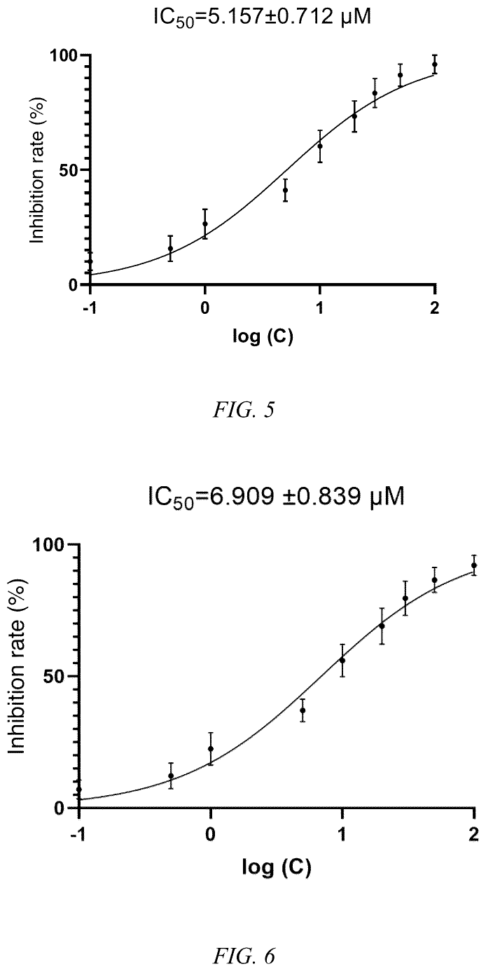

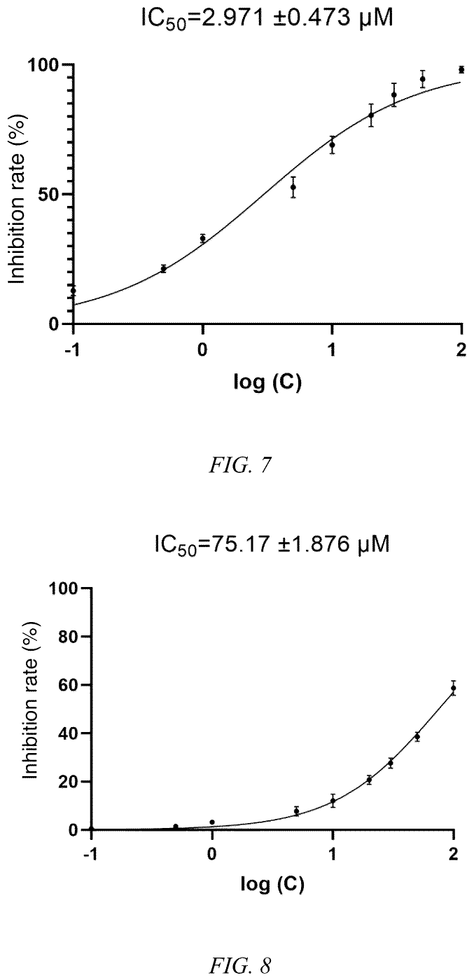

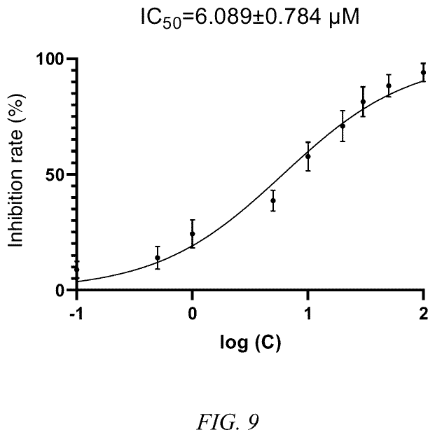

Experimental results are shown in Table 2, the gossypol, (−)-gossypol, (+)-gossypol, and acetate gossypol in this embodiment all have inhibitory activity on SARS-CoV-2 3CL proteases. The gossypol, (−)-gossypol, and acetate gossypol have a stronger inhibitory effect on SARS-CoV-2 3CL proteases, and the IC₅₀ values are all below 10 μM. (FIG. 4 to FIG. 6 respectively shown the inhibitory effects of gossypol, (−)-gossypol, (+)-gossypol, and acetate gossypol on SARS-CoV-2 3CL proteases.

TABLE 2
Inhibitory activity of gossypol, (−)- gossypol, (+)- gossypol,
and acetate gossypol on SARS-CoV-2 3CL proteases

| | Compound | | | | | | | | | | | |
|---|---|---|---|---|---|---|---|---|---|---|---|---|
| | Gossypol (IC₅₀ = | | | (−)- gossypol (IC₅₀ = | | | (+)- gossypol (IC₅₀ = | | | Acetate gossypol (IC₅₀ = | | |
| | 6.909 ± 0.839) | | | 2.971 ± 0.473) | | | 75.17 ± 1.876) | | | 6.089 ± 0.784) | | |
| Concentration/μM | 1 | 2 | 3 | 1 | 2 | 3 | 1 | 2 | 3 | 1 | 2 | 3 |
| 0.1 | 7.14 | 3.41 | 10.53 | 12.83 | 10.83 | 14.77 | 0.12 | 0.76 | 0.44 | 8.97 | 5.12 | 12.31 |
| 0.5 | 12.17 | 7.21 | 17.12 | 20.99 | 19.88 | 22.88 | 0.95 | 1.38 | 1.98 | 13.86 | 9.18 | 18.84 |
| 1 | 22.91 | 16.13 | 28.41 | 31.43 | 32.87 | 34.65 | 2.45 | 3.18 | 3.87 | 24.71 | 17.94 | 30.17 |
| 5 | 36.24 | 33.24 | 41.67 | 52.19 | 48.98 | 56.91 | 5.69 | 7.88 | 9.54 | 37.81 | 34.61 | 43.51 |
| 10 | 57.41 | 49.17 | 61.32 | 71.90 | 65.43 | 69.78 | 9.43 | 11.87 | 14.87 | 59.14 | 50.87 | 63.21 |
| 20 | 71.23 | 61.34 | 74.52 | 82.54 | 75.44 | 83.47 | 18.73 | 20.90 | 22.44 | 72.87 | 63.41 | 76.32 |
| 30 | 83.69 | 72.15 | 83.12 | 90.45 | 83.21 | 91.45 | 25.21 | 28.43 | 29.13 | 85.31 | 73.94 | 84.81 |
| 50 | 88.41 | 81.17 | 90.16 | 96.88 | 90.68 | 95.87 | 36.55 | 40.32 | 38.72 | 90.17 | 82.76 | 91.83 |
| 100 | 93.21 | 87.84 | 95.27 | 99.23 | 96.73 | 98.44 | 58.34 | 61.83 | 55.88 | 95.41 | 89.67 | 97.16 |

In summary, gossypol, (−)-gossypol, and acetate gossypol all show excellent inhibitory activity on SARS-CoV 3CL proteases and SARS-CoV-2 3CL proteases, and may be developed and researched as an anti-Coronavirus drug.

1-6. (canceled)

7. A method of treating a disease caused by a Coronavirus, comprising
providing gossypol, or optical isomers thereof; and 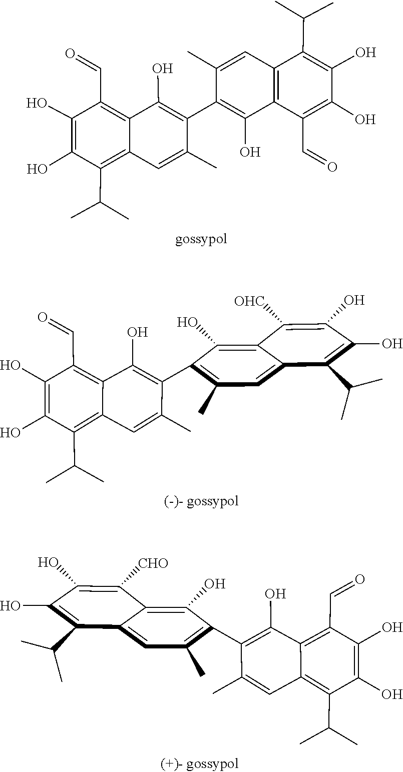
treating a subject with the disease caused by the Coronavirus with gossypol, or the optical isomers thereof.

8. The method according to claim 7, wherein the Coronavirus is MERS-CoV, SARS-CoV, or SARS-CoV-2.

9. The method according to claim 7, wherein the disease caused by the Coronavirus is an infectious disease caused by MERS-CoV, SARS-CoV, or SARS-CoV-2 or a combination thereof.

10. An anti-Coronavirus drug, comprising gossypol, or optical isomers thereof, A pharmaceutically acceptable salt thereof,

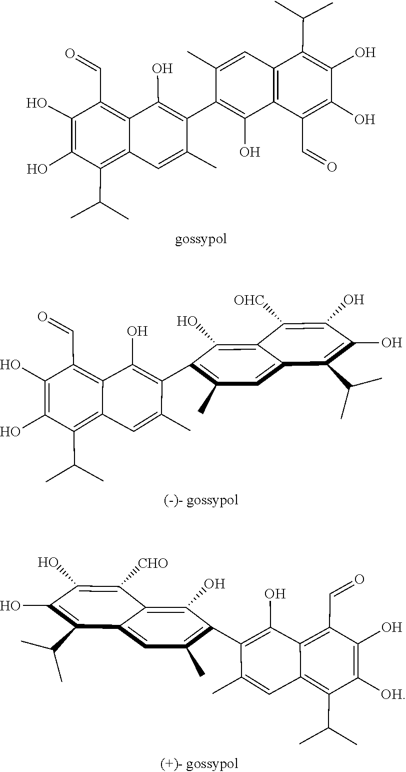

11. The anti-Coronavirus drug according to claim 10, wherein the anti-Coronavirus drug is a capsule, a tablet, a granule, a gel, a slow-release formulation, an oral solution, a dripping pill, an emulsion, an injection, or a nanoagent.
